(19) United States
(12) Patent Application Publication
(10) Pub. No.: US 2023/0047540 A1
(43) Pub. Date: Feb. 16, 2023

(54) TOILET SEAT COMPRISING A DEVICE FOR DETECTING VALUES

(71) Applicant: Martin HERBST, Österreich (AT)

(72) Inventor: Martin HERBST, Österreich (AT)

(21) Appl. No.: 17/796,836

(22) PCT Filed: Jan. 27, 2021
(86) PCT No.: PCT/EP2021/051805

§ 371 Date: Aug. 1, 2022

(30) Foreign Application Priority Data
Feb. 5, 2020 (EP) 20155560.4

(51) Int. Cl. A61B 5/00; A47K 13/24; A61B 5/0531; A61B 5/0537

(52) U.S. Cl. CPC A61B 5/0075; A47K 13/24; A61B 5/0531; A61B 5/0537; A61B 5/4836; A61B 5/486; A61B 5/6891; A61B 5/7445

(57) ABSTRACT

Toilet seat comprising a device for detecting values, in particular health-related values of a user, wherein the device for detecting values comprises a multispectral camera for collecting multispectral data, system for detecting values comprising such a toilet seat, and method for detecting values of a user.

The invention relates to a toilet seat comprising a device for measuring values, a system for detecting values comprising such a toilet seat, as well as to a method for detecting values of a user by way of such a toilet seat or such a system.

Toilet seats are used by everyone several times a day. It is known, for example from EP 3 417 766 A1, to equip such toilet seats with weight sensors, sensors for measuring the fat content, the water content, and the protein content of tissue in order to determine body values of a user. The respective values can then be verified each time the user uses the toilet so that regular verification of the values is ensured without the user having to take additional steps and incur effort to carry out the measurements. The data collected can be used to monitor the physical condition and fitness of the user. A single toilet seat can also normally be used by several and basically any number of users.

Proceeding from there, the object now arises to specify an improved toilet seat which enables, in particular, more precise and/or cost-effective determination of the state of health of the user.

This object is satisfied by a toilet seat according to claim 1, a system according to claim 10, and a method according to claim 14. Further embodiments are described in the dependent claims.

A toilet seat comprises a device for detecting values, in particular health-related values of a user. The device for detecting values comprises a multispectral camera for collecting multispectral data.

Health-related values of a user in this text are values which each by itself and these values collectively allow for conclusions to be drawn relating to the state of health of a user. These health-related values can comprise or be values that are not measured directly in or on the body of a user, but allow for conclusions to be drawn about the body of a user, for example, measured values of excretions, waste materials, odors, hair and/or nails. Health-related values can alternatively or additionally comprise or be values that are measured directly on the body of a user, for example, body parameters such as temperature, heart rate, body fat content, body protein content, body water content, blood oxygen saturation, and/or other body parameters.

Multispectral cameras can provide multispectral data by capturing light or radiation in different spectral ranges (i.e. light or radiation in different frequency ranges). With the information in different spectral ranges (frequency ranges), such multispectral data can provide information about constituents and existing molecules in a material from which emanating light was captured by the multispectral camera. An analysis of the substances detected can therefore be carried out with such a multispectral camera. A multispectral camera can collect, for example, multispectral data in at least two, for example, two, three, or four, spectral ranges, where each spectral range can comprise a continuous spectrum, e.g. of at least 20 nm or at least 50 nm.

A multispectral camera can focus light and then collect multispectral data from the totality of the focused light, or collect multispectral data in a two- or three-dimensional image, where multispectral data can be collected for each point (or other predefined region) of the two- or three-dimensional image

In particular, the multispectral camera can collect multispectral data (and thereby create it for further use). This multispectral data can contain information about reflected or emitted electromagnetic radiation at different wavelengths. The multispectral camera can capture different spectra, for example, the complete visible light spectrum (or partial range of the visible light spectrum) and/or non-visible light spectra, e.g. ultraviolet or infrared, and/or spectra that reach beyond ultraviolet or infrared spectra. For example, the multispectral camera can detect (and measure) the amount of light reflected and/or absorbed by a wide variety of substances, such as bacteria or molecules, so that they can subsequently be analyzed. The multispectral camera can comprise one or more sensor(s) which can capture the light reflected or emitted by the substances at different wavelengths.

With the multispectral camera, a conventional (two-dimensional) image can optionally be recorded in addition to the multispectral data. Such an image can then be evaluated, e.g. by automated image analysis. For example, the appearance of excretions can be taken into account. Alternatively, one, two, or more additional cameras, which can record a conventional (two-dimensional) image, can be comprised in the device for detecting values.

With regard to the spectra to be captured (in particular with regard to the frequencies used), the multispectral camera can be matched to the materials being examined, in particular to the molecules to be detected. The multispectral camera can be configured such that the spectral ranges it captures are those spectral ranges in which the molecules to be detected or the materials examined have typical spectra. Molecules to be detected can be or comprise, for example, blood components, blood, components of urine, components of the skin or the like. Early detection of warning signs such as acidification of the body or signs of inflammation and/or exposure to environmental toxins, e.g. heavy metals or drug residues, and/or early detection of diseases such as colon cancer or other forms of cancer can then take place.

The spectra of the multispectral camera can be adjusted by mechanical and/or electronic filters such that the spectra recorded can be used to draw conclusions about the presence or absence of the materials examined or molecules to be detected and/or quantitative determination of the amount of the molecules to be detected can be done. Such a multispectral camera can be controllable with regard to the spectra (frequencies) used and/or can be used with different filters.

The multispectral camera can comprise a (mechanical or electronic) filter that removes the spectrum of water so that this (main) component of the body and the body's own substances do not appear as an interference signal.

The multispectral camera can be arranged to be movable in the toilet seat so that the region covered by it (i.e. the region from which light can fall onto the multispectral camera) can be altered. In other embodiments, it is arranged to be stationary in the toilet seat so that it can record a predetermined region (i.e. light therefrom).

It can operate with the ambient light or light sources present in the toilet or at other places. Alternatively, a light source integrated into the multispectral camera or separate thereform can be comprised in the toilet seat for the multispectral camera. A light source comprised in the toilet seat (integrated into the multispectral camera or separate therefrom) can in particular cover one or more spectral range(s), for example, infrared, UV and/or spectral light (e.g. visible light or a partial range of visible light). The spectrum of a light source comprised in the toilet seat can be adjustable (selectable) by controlling the light source. Alternatively, one or more light sources for specific spectral range(s) can be comprised in the toilet seat. The light source can be arranged in the toilet seat, but can also be arranged separately at the toilet. The light source can be fixedly arranged or, alternatively, the light source can be movable so that the direction of the light it emits can be predefined.

Such a toilet seat can be equipped with a standard connection so that it can be retrofitted to commercially available toilets.

The toilet seat can comprise a rechargeable battery for supplying the device for detecting values. Alternatively or additionally, it can comprise a power connection and/or other option for supplying power to the device for detecting values comprised therein, e.g. by charging a rechargeable battery. For example, it can comprise a charging port such as a USB, mini-USB, or other charging port and/or a conventional power connection that can be connected to the mains. Alternatively, the toilet seat can be configured such that a rechargeable battery in the toilet seat can be charged by induction. Such a toilet seat can also be delivered with a matching toilet or can be provided for a matching toilet. In such a case, the toilet seat can be supplied with power, for example, by way of a power connection of the toilet and, for example, the power can be supplied to the toilet seat by way of the connecting members to the toilet.

Furthermore, the toilet seat can enable data, in particular detected values, to be transmitted to a processor exterior to the toilet seat, for example, via a cable, such as a USB cable or network cable and/or via a wireless connection, for example, WI Fl or Bluetooth.

A toilet seat according to the invention can comprise a toilet lid. The toilet lid can be arranged on the toilet seat and protect it when not in use. The toilet lid or the toilet seat can comprise a device for cleaning the toilet seat so that the toilet seat comprising the device for detecting values or parts thereof can be cleaned after each use and made available for subsequent use.

The multispectral camera can be arranged in the toilet seat behind a window. It can therefore be visible from the outside through the window. The window can be light-transmissive in the spectral ranges in which the molecules to be detected have typical spectra, i.e. let at least 50%, e.g. at least 80% of the light pass through in the spectral ranges under consideration. A light source can be placed behind the same as or a different window than the multispectral camera. A light source can also be arranged without a window.

Such a window in the toilet seat can be acid resistant so that it can be cleaned with commercially available toilet cleaning chemicals. It can align flush with the toilet seat so that there is no space between the toilet seat and the window where dirt can collect.

In a toilet seat, the multispectral camera can be oriented to collect multispectral data of excretions. In particular, the multispectral camera can be oriented such that multispectral data of the contents of the toilet bowl can be collected. For example, the multispectral camera can be arranged on the underside of the toilet seat and/or on the inner side of the toilet seat. When specifying the upper side, underside, inner side and outer side of the toilet seat, it is assumed that the toilet seat is in a state ready for use, i.e. resting on the toilet, with the upper side facing the user, the underside facing the toilet bowl, the outer side is arranged on the side of the toilet seat facing away from the toilet and the inner side of the toilet seat defines the opening through which the toilet can be used. The toilet seat can be configured such that it is suitable for resting with the underside on the toilet bowl and protruding slightly inwardly over the edge of the toilet bowl, i.e. in particular not flush therewith, for example, to project at least 0.5 cm, for example, at least 1 cm or at least 2 cm inwardly beyond the rim of the toilet bowl.

The multispectral camera can be arranged in particular on the underside of the toilet seat, e.g. in a region which borders the inner side of the toilet seat. With such an arrangement of the multispectral camera in a toilet seat that is configured to project inwardly beyond the rim of the toilet bowl, it can be achieved that the multispectral camera is not hidden behind the rim of the toilet bowl.

A multispectral camera, which is or can be oriented to collect multispectral data of excretions, can be or is matched to the molecules or materials to be examined, in particular with regard to the spectra to be detected. Such materials and molecules to be examined can comprise, for example, blood, blood components, cancer cells or their residues, color and/or other residues in excretions The multispectral camera can be configured in particular to examine spectral ranges in which the respective molecules or materials have typical spectra, e.g. through one or more filters. The use of a multispectral camera which can be oriented to collect multispectral data from excretions has the advantage that—unlike conventional methods using test strips or laboratory analysis—no consumables have to be disposed of, although excretions can be examined comprehensively.

The multispectral camera can be oriented to collect multispectral data of the body of a user. For example, it can be placed at the upper side of the toilet seat (optionally behind a window) so that multispectral data of the body of a user can be collected while seated on the toilet seat. In such a case, the light source can optionally be comprised in the camera and/or can be arranged behind the same window as the camera.

Alternatively, the multispectral camera that is oriented or can be oriented to collect multispectral data of the body of a user can be arranged on the inner side of the toilet seat so that the data of the body of a user can be collected by recording at an angle upwardly while the user is sitting on the toilet seat. When arranged on the inner side of the toilet seat, the multispectral camera can be arranged to be movable (and controllable) so that multispectral data of the excretions as well as multispectral data of the body of a user can be collected with one and the same multispectral camera. This multispectral camera can also be adaptable with regard to the spectra used, for example, be controllable with electronic and/or mechanical filters so that it can be adjusted for different materials to be examined and molecules to be detected.

The multispectral camera can be aligned in particular such that multispectral data of the skin can be collected and through the skin of the user, for example, multispectral data that describe the skin color and skin texture, or the oxygen saturation of the blood or the like. Such a multispectral camera, which is or can be configured to collect multispectral data of the body of a user, can be matched in terms of the materials and molecules to be examined, for example, to the spectra that allow for conclusions to be drawn about oxygen saturation or other components of the blood or the skin.

In the toilet seat, the device for detecting can comprise a first and a second multispectral camera for collecting multispectral data. Each of these two multispectral cameras can be configured as previously described. For example, one or both multispectral cameras (individually or together) can be arranged behind a window. The first multispectral camera can be oriented to collect multispectral data of the excretions and be configured as previously described for a multispectral camera for collecting multispectral data of the excretions. The second multispectral camera can be oriented to collect multispectral data of the body of a user and can be configured and arranged as described above for such a multispectral camera.

The device for detecting values of a user can comprise a sensor for detecting the skin color of a user, for example, a spectral sensor which is suitable for determining the skin color of the user. This information can then be used to adjust the intensity and spectral emission of a light source for further detecting values of a user with the multispectral camera.

The device for detecting values of a user can alternatively or additionally comprise a skin surface detector. A skin surface detector can be configured, for example, to measure particles and odors on the skin, for example, to measure the electrodermal activity of the skin and/or the amount of perspiration on the skin.

The device for detecting values of a user can alternatively or additionally comprise a laser spectrometer. A laser spectrometer can be used, for example, to examine atomic or molecular components and, for example, create fingerprints of molecular compositions of biological samples of any kind. Such fingerprints of molecular compositions of biological samples can allow, in particular, for determining whether certain (known) atomic or molecular components are present and, optionally, in what quantity or concentration these components are present.

The device for detecting values can comprise a gas sensor with which, for example, further information about the excretions, in particular their gas release, or gas release from the skin or gas mixtures from the ambient air can be obtained.

The gas sensor can be a chemical sensor for the detection of gaseous substances. For example, the proportion of certain chemical ingredients in the gas can be converted by the chemical sensor into an electrical signal that can be read out (and therefore detected as a value that can be taken into account, for example, when determining one or more parameters for the state of health of the user). The gas sensor can have limitations in the selectivity and response to one or more target substances. The sensor selectivity can be preset, for example, with a specific reactivity to one, two, or more target substances (where the sensor, for example, selectively detects one, two, or more target substances and can show no (relevant) response to other substances).

Alternatively, the sensor sensitivity can be varied (e.g. by controlling the sensor). The measurements can be carried out, for example, by electrical and/or mechanical sensors. The gas sensor can optionally be a molecular sensor. The gas sensor can be an active or a passive sensor.

The gas sensor can output a CO₂eq (CO₂ equivalent) which can mathematically map conclusions drawn from the H2 concentration.

The device for detecting values can alternatively or additionally comprise one or more further sensors, for example, one or more weight sensors, measuring sensors for measuring skin resistance (for example for determining body fat percentage), a sensor for measuring the water content in the tissue, a sensor for measuring the bone mass, a pollutant sensor, a high-frequency sensor or sensors for other frequencies, an X-ray sensor, a sensor or a sensor system for analyzing the autonomic nervous system, a temperature sensor, a sensor for measuring the heartbeat and/or a measuring sensor for blood pressure, ECG, a sensor for measuring blood sugar, a sensor for measuring blood flow and/or blood volume, a sensor for measuring lactate and/or salt concentration in the blood. The device can comprise sensors or gauges comprising a Geiger counter or other radiation sensor for measuring radioactivity in the excretions and/or the body of the user. Each such sensor can contribute to enabling gaining an even more accurate picture of the health status of a user. The device for detecting values can also comprise a biofeedback sensor and/or a sensor and/or a sensor system for bio resonance measurement.

The toilet seat can comprise a display that enables displaying information, for example, simple information or warnings and/or biofeedback.

The invention further comprises a system for detecting values of a user which comprises a toilet seat as described above and a processor or a processor system composed of two or more processors, where the processor or the processor system is configured to use multispectral data to determined one or more parameters on the state of health of the user and to output a result (the processor or the processor system are hereafter in their entirety also referred to as an evaluation processor).

The processor or a processor of the processor system can be arranged in the toilet seat. The processor in the toilet seat can be configured to detect the measured values of the device for detecting values, to store them temporarily, and to transfer them to the outside, for example, by way of a cable or in a wireless manner.

The processor or one or more processors of the processor system can alternatively or additionally be arranged outside the toilet seat, e.g. in a nearby computer, in the cloud, in a cloud system, a decentralized system, a smartphone or other mobile device.

The one or more parameters relating to the state of health of the user can be in particular the multispectral data or a measured value directly or a value calculated from the multispectral data and optionally further measured values or a value that is derived in some other way. In particular, information can be obtained from the multispectral data from one or more multispectral cameras for determining the one or more parameters relating to the state of health of the user. For example, the evaluation processor can be configured to algorithmically determine information from the multispectral data, e.g. about one (or more) component(s) of materials examined and/or about molecules to be detected. For this purpose, it can be configured in particular to eliminate background spectra (e.g. water and/or reflections from the toilet bowl) or other interference signals from the multispectral data and/or to obtain information about the presence or absence of certain component(s) and/or molecules to be detected by inspecting and comparing the multispectral data in certain spectral ranges. This information about one or more component(s) of materials examined and/or about molecules to be detected can represent one or more parameters relating to the state of health of the user.

To determine the one or more parameters relating to the state of health of the user, information described above from the multispectral data (e.g. about one or more component(s) of materials examined and/or about molecules to be detected) can alternatively or additionally be combined with one or more further pieces of measurement data optionally available from one or more other sensors and/or with optionally available one or more pieces of information about the user, e.g. age, size, medical data or the like. One or more health parameters can be determined thereform, e.g. by calculation or comparison with standard values. They can optionally be compared with the user's data from the past in order to determine a trend. The evaluation processor can be configured to perform one, several, or all of the steps previously described with regard to the evaluation.

The evaluation processor is configured to output a result. The result can be, comprise, or be based on one or more health parameters relating to the state of health of the user.

The result can be displayed on a controllable display connected to the evaluation processor, e.g. a monitor, be sent to one or more recipients in text or machine-readable form, be output by voice output or be sent to another processor, e.g. the processor in the toilet seat or a smartphone, or be output in another manner. For example, a result can be shown on the display of the toilet seat and/or on another device, e.g. the smartphone of the user.

For example, the evaluation processor can be configured to provide an overall binary result based on the values which either indicates that everything is OK or that the values measured suggest a problem. Alternatively, the evaluation processor can be configured to provide a more detailed result, for example, several parameters relating to the state of health of the user, a comparison with data of the user from the past, a trend with regard to the data, a biognostic value of the user or the excretions, a comparison with normal values, and/or or to output suggestions for the user, e.g. nutritional advice or the like. In addition, the result can be stored by the evaluation processor and taken into account when determining future results, and/or further steps can be carried out based on the result.

On the basis of data supplied, the evaluation processor can be configured, in particular, to perform machine learning by way of one or more algorithms based on data supplied, in particular a training data set with detected values that comprise multispectral data, and optionally further information, for example, medical information about the individuals, for example, age, size, weight, preconditions, or the like, the detected values of which are supplied. Algorithms can therefore be developed that calculate one or more parameters relating to the state of health of the user according to the current state of knowledge. Such algorithms can take into account in particular quantum physics.

The evaluation processor can be configured to carry out further machine learning, optionally after initialization of the system based on additional or new health information and/or with further training data sets, so that the system can be further developed. In particular, new medical findings can be taken into account in the system by machine learning. In addition, the evaluation processor can be configured to carry out further machine learning steps at another point in time (e.g. with each additional determination of one or more parameters relating to the state of health of the user or after a certain number of cycles of determining one or more parameters relating to the state of health of the user). The artificial intelligence of the evaluation processor can thus be further developed by machine learning.

In addition to the toilet seat and the evaluation processor, the system can also comprise one or more further displays. For example, the system can comprise a display (e.g. a monitor) on which the result can be displayed and which can be installed, for example, next to the toilet, or comprise the display of a mobile terminal device, the processor of which is the evaluation processor of the system. In particular, the evaluation processor can be a system of several processors in which, for example, some processors can be located in a cloud which, for example, can carry out the computationally intensive steps, whereas a processor in the toilet seat or in a mobile terminal device or the user's smartphone outputs and optionally displays the one or more health parameters of the user that were calculated.

The evaluation processor can be configured to detect the skin color of the user and, based thereupon, to determine the spectrum and/or the intensity of the light source for a multispectral camera and to control the light source accordingly so that the detection of values can then be carried out with the corresponding spectrum and/or the corresponding intensity of the light source, for example, by switching on suitable filters for the light source and/or adjusting the intensity of the light source. The user's skin color can be detected, for example, by way of sensors for detecting the user's skin color or by way of a first measurement with the multispectral camera. It can be advantageous to detect the user's skin color before the actual measurements are carried out on the body of the user, since correspondingly different intensities of light sources and/or different spectra may be required in dependence of the pigmentation of the user's skin for obtaining measurement values through the skin of the user, for example, collecting information about the blood in the body of the user. In addition, information about the skin color of the user can make it possible to eliminate the skin color of the user accordingly from the spectra that are obtained through the skin.

The evaluation processor can also be configured to eliminate interference signals, such as one or more spectra, e.g. the spectrum of water, the spectrum of one or more windows behind which the light source and/or the multispectral camera are arranged, and/or the spectrum of the background, e.g. reflections of the toilet bowl, from the multispectral data (and to thus determine the one or more parameters relating to the state of health of the user without these interference signals).

The evaluation processor can be configured to perform a treatment step based on the result. For example, it can be configured to perform bio resonance therapy based on the result of the sensor system for bio resonance measurement or to provide bio feedback.

The invention further comprises a method for detecting values of a user by way of a toilet seat comprising a device for detecting values as previously described and/or a system for detecting values of a user as previously described. In such a method, one or more parameters relating to the state of health of a user are determined using multispectral data and a result is output. All the steps that have been described above and that can be carried out in the system or with the toilet seat, respectively, in particular by the evaluation processor, can accordingly be steps of the method.

Further aspects of the invention shall be explained with reference to the figures, where

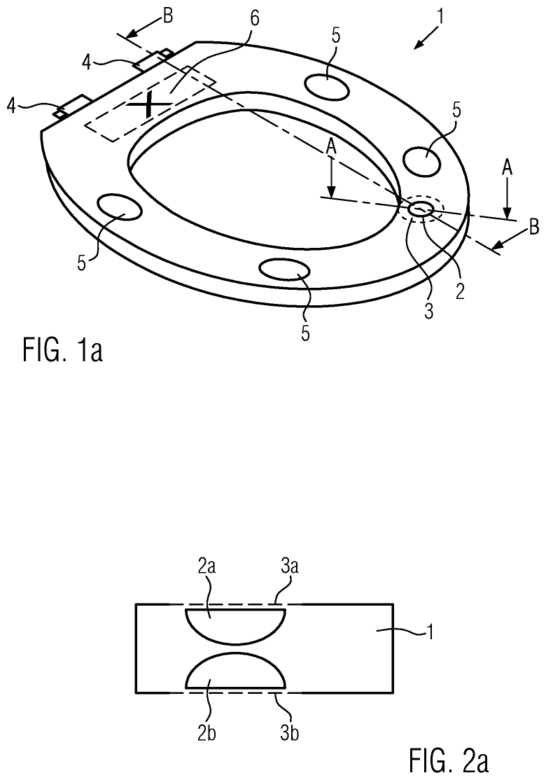

FIGS. 1a and 1b show a toilet seat viewed at an angle from above;

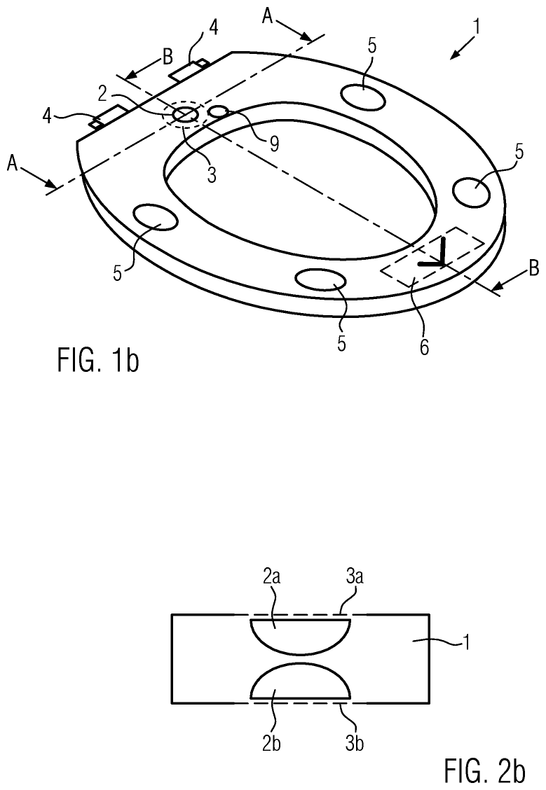

FIGS. 2a and 2b show a cross section through part of a toilet seat;

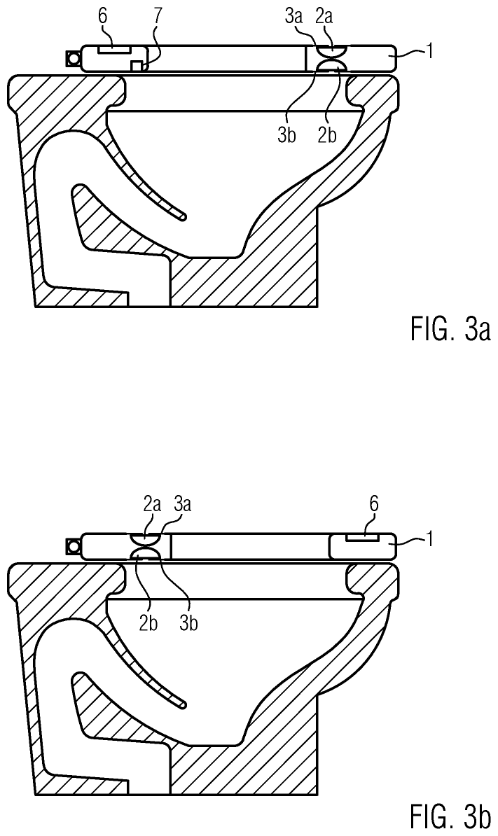

FIGS. 3a and 3b show a cross section through a toilet bowl with the toilet seat;

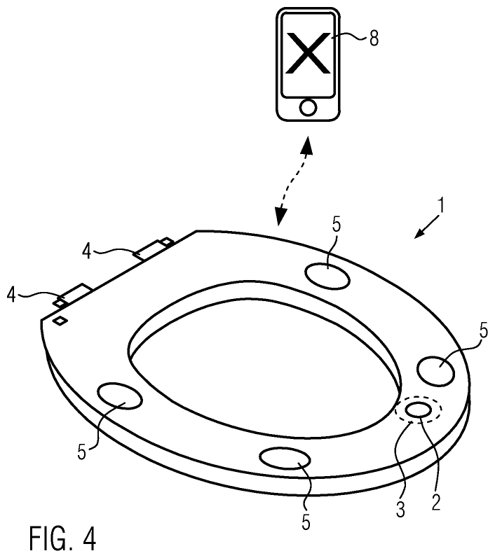

FIG. 4 shows a system comprising a toilet seat.

FIG. 1a show a toilet seat at an angle from above. Toilet seat 1 comprises in particular a multispectral camera 2 for collecting multispectral data (in a device for detecting values). In the example shown, this multispectral camera 2 is arranged behind a window 3 in the toilet seat and is directed such that it is oriented to capture a spectrum of the body of the user. Multispectral camera 2 is arranged in toilet seat 1 in particular such that it is typically disposed below the leg of a user when the user is sitting on toilet seat 1 in a normal posture.

The toilet seat can comprise a connection 4 for connecting the toilet seat to a toilet bowl, where this can be a standard connection typically used for normal toilets so that the toilet seat can be connected to any commercially available toilet. Alternatively, the connections can be configured to match a specific toilet, which can be sold, for example, with the toilet seat so that, for example, power supply and/or data transmission can also be possible by way of connections 4 (presently not shown). The toilet seat can comprise further sensors 5, for example, one or more of the following sensors: a gas sensor, a sensor for detecting skin color, a skin surface detector, a weight sensor, a temperature sensor, ECG, a Geiger counter, a bio feedback sensor, a sensor system for bio resonance measurement. Toilet seat 1 can additionally or alternatively optionally comprise a laser spectrometer (not shown).

As shown, toilet seat 1 can optionally comprise a display 6. In the figure, display 6 is arranged, for example, at the rear end of toilet seat 1 so that the user can see this when rising and flushing the toilet. Also by way of example, a binary result in the form of an X is shown on display 6 which can indicate one or more parameters relating to the state of health of the user deviating from the recommended parameters. Of course, other results, such as a different form of warning, a binary o.k. in the form of a tick or any other form, or a more detailed result, for example, in the form of one or more parameters, measured values, or pieces of information can also be displayed on the display.

In addition or as an alternative to the multispectral camera shown, the toilet seat can optionally comprise a multispectral camera which can be directed at the excretions (not visible in FIG. 1a). In other embodiments, the toilet seat can comprise a multispectral camera which can be directed at the body of the user and the excretions and which is arranged to be movable and controllable, e.g. on the inner side of toilet seat 1 (not shown in FIG. 1a).

FIG. 1b shows an alternative embodiment of a toilet seat at an angle from above. In this embodiment, a multispectral camera 2 is arranged centrally with respect to axis B on the inner side of the rear region of toilet seat 1, and a display 6 is arranged centrally with respect to axis B in the front region of toilet seat 1. Axis B presently describes the axis of symmetry of the shape of the toilet seat (where the device for detecting values, in particular multispectral cameras, sensors, and also the display etc. are not taken into account with regard to the axis of symmetry of the shape of the toilet seat, i.e. they need not be arranged symmetrically with regard to the axis of symmetry). In the embodiment shown, multispectral camera 2 is arranged such that the user does not normally come to sit on multispectral camera 2 when using the toilet seat.

In other embodiments, multispectral camera 2 and/or display 6 can also be arranged not centrally with respect to axis B, but laterally offset with respect to axis B (not shown).

As shown, toilet seat 1 can also comprise a connection 4, which can be configured in particular as described in FIG. 1a, for connecting the toilet seat to a toilet bowl. Furthermore, toilet seat 1 can comprise one or more further sensors 5, for example, such sensors as described for FIG. 1a. Toilet seat 1 can optionally comprise a laser spectrometer. Toilet seat 1 can optionally comprise a gas sensor 9 which can be arranged, for example, in the rear half, e.g. in the rear third, of toilet seat 1. Gas sensor 9 can be arranged, for example, on the inner side of toilet seat 1 or closer to the inner side of toilet seat 1 than to the outer side of the toilet seat.

A binary result in the form of a tick is shown by way of example on display 6 and can indicate that the one or more parameters relating to the state of health of the user are within the predefined (normal) parameters. Other results, for example, as described for FIG. 1a, can of course also be displayed on display 6. As described for FIG. 1a, a multispectral camera can alternatively or additionally be comprised, e.g. a multispectral camera directed at the excretions (not shown in FIG. 1b). In other embodiments, a multispectral camera can be comprised in the toilet seat and can be directed at the body of the user and the excretions and which is arranged to be movable and controllable, e.g. on the inner side of toilet seat 1 (not shown in FIG. 1b).

FIG. 2a shows a cross section through a part of a toilet seat 1 according to the invention, as it could appear, for example, through line A in the toilet seat of FIG. 1a. Shown there is a first spectral camera 2a behind a window 3a and a second multispectral camera 2b behind a window 3b.

Multispectral camera 2a is arranged such that it is typically directed at the body of the user (there typically the underside of a thigh of the user) when using the toilet seat. A light source can be integrated into multispectral camera 2a shown. The spectrum of the light source can comprise spectra that are suitable for examining a body of the user, in particular, for example, infrared radiation, UV radiation, or other spectral ranges that allow for conclusions to be drawn about body components, e.g. blood components or blood oxygen saturation. In other embodiments, the light source can be placed behind same window 3a as multispectral camera 2a, but separated thereform (not shown).

Second multispectral camera 2b is, for example, behind window 3b and oriented downwardly so that it is directed in the direction towards the toilet bowl when the toilet seat is used (and can therefore take pictures of the excretions in the toilet bowl). This multispectral camera can also comprise an integrated light source or use a separate light source. Alternatively or additionally, it can be configured for use with light that is present anyway, e.g., ambient light or light from an illuminated toilet.

In cross section radially through the toilet seat and multispectral camera 2b, window 3b can be arranged closer to the inner side of the toilet seat than to the outer side—as shown in FIG. 2—so that multispectral camera 2b is not covered by the rim of the toilet bowl when the toilet seat is used. In cross section radially through the toilet seat and multispectral camera 2b, window 3a in FIG. 2 is also arranged offset toward the inner side of the toilet seat. However, this arrangement can be different in other embodiments, for example, multispectral camera 2a can be arranged in cross section through the toilet seat and multispectral camera 2a centrally between the inner side and the outer side of toilet seat 1 or closer to the outer side of toilet seat 1.

FIG. 2b shows a cross section through a part of a toilet seat 1 as it could appear, for example, through line A in the toilet seat of FIG. 1b. Shown there is a first spectral camera 2a behind an optional window 3a as well as a second multispectral camera 2b behind an optional window 3b.

Multispectral cameras 2a and/or 2b can each be configured as described for multispectral cameras 2a and/or 2b in FIG. 2a. Alternatively or additionally (not described in FIG. 2a), multispectral camera 2a can be configured for use with light that is present anyway, e.g. with ambient light.

FIG. 3a shows a cross section through multispectral cameras 2 and toilet seat 1 while the latter is arranged on a toilet bowl. Such a cross section can be, for example, a cross section along line B in FIG. 1a. Two multispectral cameras 2a and 2b are drawn in there by way of example, where one multispectral camera 2a optionally behind window 3a is directed at the body of the user and second multispectral camera 2b optionally behind window 3b is directed at the excretions. In the example shown in FIG. 3a, a light source 7 is optionally provided on a rear part of toilet seat 1 for providing light for multispectral camera 3b. Light source 7 can comprise spectra that can be adjusted, e.g. by mechanical or electronic filters, and/or comprise a spectral distribution of the light that is matched for examining excretions. Light source 7 can be a point light source or be expanded, e.g. configured as a light strip. A light strip-shaped light source 7 can run, for example, parallel to the inner side of the toilet seat and therealong for more than 1 cm, for example, at least 10 cm. Also shown in FIG. 3a is an optionally present display 6 of toilet seat 1 which can be configured, for example, like the display in FIG. 1a.

FIG. 3b shows a cross section through multispectral cameras 2a and 2b and toilet seat 1 while the latter is arranged on a toilet bowl. Such a cross section can be, for example, a cross section along line B (axis B) in FIG. 1b. Multispectral cameras 2a can optionally be arranged behind window 3a and configured as described for multispectral cameras 2a in FIG. 3a. Multispectral camera 2b can optionally be arranged behind window 3b and configured as described for multispectral cameras 3b in FIG. 3a. A light source 7 can optionally be comprised in toilet seat 1 and can be configured, for example, as described for light source 7 in FIG. 3a (not shown).

FIG. 4 shows a system comprising a toilet seat 1. Comprised in the system shown in FIG. 4 is a toilet seat 1 in which the device for detecting values can comprise a multispectral camera 2 behind an optional window 3 and optionally one or more sensors 5 and/or a laser spectrometer. In addition, the system also comprises an external display 8 which is presently shown in the form of a mobile terminal device, e.g. a smartphone, and at which a result can be output from the evaluation processor. In other embodiments, the system can comprise a different display, e.g., a different monitor (not shown in FIG. 4). In other embodiments, multispectral camera 2 (or several multispectral cameras, e.g. multispectral cameras 2a, 2b) can be arranged at different locations in the system in toilet seat 1, e.g. arranged as shown in FIG. 1b or FIG. 2b. Alternatively or additionally, the system can comprise a display in the toilet seat (not shown).

The evaluation processor in such a system can comprise, for example, the processor of the mobile terminal device, e.g. of the smartphone, which is configured by an app to output a result.

In some embodiments, the evaluation processor can optionally also comprise one or more further processors, e.g. located in the cloud, which are configured to carry out further steps, e.g. using multispectral data to determine one or more parameters relating to the state of health of the user. In other embodiments, these further steps can also be carried out only by the processor of the mobile end device, e.g. of the smartphone. For data protection reasons, it can namely be advantageous to have the values detected, in particular multispectral data and optional further data collected by one or more sensors, be processed directly in a mobile device (comprised in the system) that can only be physically accessed, for example, by the user or a small group of people, and that the result can be output there.

In addition, toilet seat 1 can optionally comprise a processor which is configured to use the device for detecting values to collect multispectral data and optionally further values, e.g. using sensors 5. In some embodiments, the evaluation processor can therefore be arranged only in the mobile terminal device and optionally the toilet seat, while in other embodiments, it can also comprise processors located in the cloud or in an external data center.

1. Toilet seat comprising a device for detecting values, in particular health-related values of a user, wherein said device for detecting values comprises a multispectral camera for collecting multispectral data.

2. Toilet seat according to claim 1, wherein said multispectral camera is arranged in said toilet seat behind a window.

3. Toilet seat according to claim 1, wherein said multispectral camera is oriented or can be oriented to collect multispectral data of the excretions.

4. Toilet seat according to claim 1, wherein said multispectral camera is oriented or can be oriented to collect multispectral data of the body of the user.

5. Toilet seat according to claim 1, wherein said device for detecting values comprises a second multispectral camera for collecting multispectral data.

6. Toilet seat according to claim 1, wherein said device for detecting values comprises a sensor for detecting the skin color of a user and/or a skin surface detector and/or a laser spectrometer.

7. Toilet seat according to claim 1, wherein said device for detecting values comprises a gas sensor.

8. Toilet seat according to claim 1, wherein said device for detecting values comprises a bio feedback sensor and/or a sensor or a sensor system for bio resonance measurement.

9. Toilet seat according to claim 1, wherein said toilet seat comprises a display.

10. System for detecting values, in particular health-related values of a user, comprising a toilet seat with a device for detecting values according to one of the preceding claims and a processor or a processor system, wherein said processor or said processor system is configured to determine one or more parameters relating to the state of health of the user using multispectral data and to output a result.

11. System according to claim 10, wherein said processor or said processor system is configured to detect the skin color of the user and based thereupon to determine the spectrum and/or the intensity of the light source for a multispectral camera and to control said light source accordingly.

12. System according to claim 10, wherein said processor or said processor system is configured to carry out machine learning by way of an algorithm on the basis of supplied data, e.g. a training data set comprising multispectral data.

13. System according to claim 10, wherein said processor or said processor system is configured to carry out a treatment step, e.g. bio resonance therapy, based on said result.

14. Method for detecting values of a user by way of a toilet seat comprising a device for detecting values according to claim 1, wherein one or more parameters relating to the state of health of a user is/are determined using multispectral data and a result is output.

15. Toilet seat according to claim 2, wherein said multispectral camera is oriented or can be oriented to collect multispectral data of the excretions.

16. Toilet seat according to claim 15, wherein said multispectral camera is oriented or can be oriented to collect multispectral data of the body of the user.

17. Toilet seat according to claim 16, wherein said device for detecting values comprises a second multispectral camera for collecting multispectral data.

18. Toilet seat according to claim 17, wherein said device for detecting values comprises a sensor for detecting the skin color of a user and/or a skin surface detector and/or a laser spectrometer.

19. Toilet seat according to claim 18, wherein said device for detecting values comprises a gas sensor.

20. Toilet seat according to claim 19, wherein said device for detecting values comprises a bio feedback sensor and/or a sensor or a sensor system for bio resonance measurement.
